(19) United States
(12) Patent Application Publication
(10) Pub. No.: US 2007/0190028 A1
(43) Pub. Date: Aug. 16, 2007

(54) METHOD AND APPARATUS FOR HEAT OR ELECTROMAGNETIC CONTROL OF GENE EXPRESSION

(76) Inventors: Jihong Qu, Maple Grove, MN (US); Craig Stolen, New Brighton, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/276,077

(22) Filed: Feb. 13, 2006

(51) Int. Cl. A61K 48/00; A61N 1/365

(52) U.S. Cl. 424/93.2; 607/4; 514/44

(57) ABSTRACT

A gene regulatory system controls biomarker, gene therapy or endogenous gene expression by emitting one or more forms of energy that regulate gene expression. The system may include a sensor to sense a signal indicative of a need for therapy. The regulation of gene expression is controlled based on the sensed signal and/or a user command. In one embodiment, the system delivers one or more electrical therapies in conjunction with controlling gene therapy or endogenous gene expression.

### CROSS-REFERENCE TO RELATED APPLICATIONS

This application is related to co-pending, commonly assigned U.S. patent application Ser. No. 10/788,906, entitled “METHOD AND APPARATUS FOR DEVICE CONTROLLED GENE EXPRESSION,” filed on Feb. 27, 2004, which is hereby incorporated by reference in its entirety.

### FIELD OF THE INVENTION

This invention relates generally to therapy of living tissue including gene therapy, for example, the use of genetically modified cells or recombinant gene therapy vectors, and particularly, but not by way of limitation, to method and apparatus for regulation of gene expression in living tissue using a device generating signals that induce gene transcription.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the organs and pump it into the lungs where the blood gets oxygenated. The body's metabolic need for oxygen increases with the body's physical activity level. The pumping functions are accomplished by contractions of the myocardium (heart muscles). In a normal heart, the sinoatrial node, the heart's natural pacemaker, generates electrical impulses, known as action potentials, that propagate through an electrical conduction system to various regions of the heart to excite myocardial tissues in these regions. Coordinated delays in the propagations of the action potentials in a normal electrical conduction system cause the various regions of the heart to contract in synchrony such that the pumping functions are performed efficiently.

“Heart attacks” or myocardial infarctions occur when there is a loss of proper blood flow to the heart. When heart tissue does not get adequate oxygen, there is a high probability that heart muscle cells will die. The severity of a myocardial infarction is measured by the amount and severity of heart damage and loss of function.

Despite advances in the treatment of myocardial infarction, patients suffer decreased quality of life due to the damage caused by the heart attack. One such damage is chronic heart failure arising from the myocardial infarction. The cardiac muscle cells, cardiomyocytes, which, in some circumstances, die during a myocardial infarction either cannot be regenerated naturally by the heart or cannot be regenerated in sufficient quantities to repair the damage following infarction. Depending on the severity of damage to the heart muscle, cardiac output, heart valve function, and blood pressure generating capacity can be greatly reduced. These results only exemplify some of the long-term devastating impacts of heart attacks on patients.

One way to treat damaged heart muscle cells is to provide pharmaceutical therapies in an effort to restore heart function. Such therapies may not be particularly effective if the damage to the heart is too severe, and pharmaceutical therapy is not believed to regenerate cardiomyocytes, but instead acts to block or promote certain molecular pathways that are thought to be associated with the progression of heart disease to heart failure.

Another treatment for damaged heart muscle is called “cell therapy.” Cell therapy involves the administration of endogenous, autologous and/or nonautologous cells to a patient. For example, myogenic cells can be injected into damaged cardiac tissue with the intent of replacing damaged heart muscle or improving the mechanical properties of the damaged region. However, the administration of myogenic cells does not ensure that the cells will engraft or survive, much less function and there is a need in the art for enhanced efficacy of cell therapies.

Another therapy for a variety of conditions is gene-based therapy which includes the delivery of therapeutic genes to targeted cells and in some cases, the use of regulatable systems. For gene-based therapies which require expression of sequences in vectors, a promoter is linked to the sequence to be expressed. Strong viral promoters can drive a high level of expression in a wide range of tissues and cells, however, constitutive expression may induce cellular toxicity or tolerance, or down regulation of expression through negative feedback.

What is needed is a device useful to spatially, temporally and quantitatively control expression.

### SUMMARY OF THE INVENTION

The invention provides for a device that is adapted to control the expression of exogenous genes transferred to, or endogenous genes in, a host mammal which genes include a regulatable, field sensitive transcriptional control element. In one embodiment, the expression of an open reading frame in those gene(s) in vivo prevents, inhibits or treats at least one symptom of a particular condition, while in another embodiment the expression of an open reading frame in an exogenous gene transferred to a mammal is useful in diagnostics. The present invention provides spatial, temporal and/or quantitative control of gene expression from one or more vectors with an exogenous gene and/or endogenous genes via an implantable or external device. In one embodiment, the vector or endogenous gene includes an open reading frame that encodes a gene product which is secreted (released into the extracellular environment) from cells which express the gene product. In another embodiment, the vector or endogenous gene includes an open reading frame for a gene product that increases expression of one or more other gene products in cells. Thus, the effect of the expression of the open reading frame may be direct, e.g., the expression results in a soluble factor(s), e.g., a soluble therapeutic factor(s), while in another embodiment, the effect of the expression of the open reading frame is indirect, e.g., the encoded gene product alters the expression of at least one other gene product that is therapeutic or diagnostic.

For example, an implantable or external device that emits a signal, the amount and/or strength of which regulates expression from a regulatable, field sensitive transcriptional control element, may be employed to regulate endogenous gene(s) with such a transcriptional control element operably linked to an open reading frame which encodes a desirable, e.g., therapeutic, gene product. Thus, a device of the invention is useful for conditions in which it is desirable to initiate or augment expression of a gene product of an endogenous gene which can be regulated by forms of energy such as thermal and/or electromagnetic energy, for example, c-myc, c-fos, jun, HSP27, HSP60, HSP70, HSP75, HSP78, and HSP90. In another embodiment, an implantable or external device that emits a signal, the amount, and/or strength of which regulates expression from a regulatable, field sensitive transcriptional control element, may be employed with a gene therapy vector that includes such a transcriptional control element operably linked to an open reading frame which encodes a desirable gene product (an expression cassette). In one embodiment, for conditions in which it is desirable to inhibit expression of a native gene or gene product, the gene therapy vector may include an appropriate antisense gene sequence or a mutant gene, e.g., one which encodes a dominant negative gene product, operably linked to the regulatable, field sensitive transcriptional control element. In another embodiment, for conditions in which it is desirable to initiate or augment expression of a gene or gene product, the gene therapy vector may include an appropriate gene sequence or a portion thereof (sense orientation), i.e., a portion that encodes a gene product with substantially the same activity as the full length gene product, operably linked to the regulatable, field sensitive transcriptional control element.

In one embodiment, an expression cassette of the invention is delivered to a host mammal via isolated nucleic acid, e.g., a plasmid, a recombinant virus or other nucleic acid containing complex optionally in conjunction with methods to enhance delivery such as electroporation. In another embodiment, donor cells that are genetically modified with an expression cassette of the invention are employed, e.g., genetically modified autologous cells. In one embodiment, a device of the invention is implanted in or externally applied to a host mammal before, concurrent with or after a vector containing the expression cassette, or cells containing the expression cassette, are administered to the mammal. The device of the invention includes a controller and a gene regulatory signal delivery device which emits a signal upon sensing a physiological parameter or a change in a physiological parameter, or as a result of a command. The amount and/or strength of the emitted signal induces expression of the open reading frame that is operably linked to the regulatable, field sensitive transcriptional control element.

The invention further provides a system. The system includes a gene regulatory signal delivery device that emits a regulatory signal which directly or indirectly regulates a regulatable, field sensitive transcriptional control element; and a controller coupled to the gene regulatory signal delivery device. The system may include an external device having a gene regulatory signal delivery device and a controller. Alternatively, the system may include an implantable device having a gene regulatory signal delivery device and a controller. The controller is adapted to control the emission of the regulatory signal based on at least a sensed physiological parameter or a change therein, or a command. In one embodiment, the system further includes a sensor to sense a physiological parameter or a change in a physiological parameter indicative of a predetermined condition. The sensor may be part of an implantable device having a gene regulatory signal delivery device and a controller or may be part of an external device. In one embodiment, the system includes a controller coupled to the sensor and to the gene regulatory signal delivery device, where the controller is adapted to control the emission of the regulatory signal based on at least the sensed physiological parameter or a change therein, or a command. In another embodiment, the system further includes a controller coupled to a telemetry module, the telemetry module adapted to receive an external command, and the controller adapted to control the emission of the regulatory signal based on at least the external command. In another embodiment, the system further includes a controller coupled to a programmable device, the controller adapted to control the emission of the regulatory signal based on a predetermined program executed by the programmable device.

The system can be used to regulate exogenously delivered genes or endogenous genes, thereby providing a therapeutic effect, or regulate certain molecules (biomarkers) that are expressed in a way that indicates pathophysiological states of an area of interest (e.g., an ischemic area of heart).

The invention also provides a method to control expression of an open reading frame present in cells of a mammal. The method includes providing a mammal having a system of the invention, e.g., a system which may include a sensor, for instance, to sense a physiological signal indicative of a predetermined condition, a gene regulatory signal delivery device that emits a regulatory signal which directly or indirectly regulates a regulatable, field sensitive transcriptional control element, and/or a controller coupled to at least the gene regulatory signal delivery device and optionally to the sensor. The controller is adapted to control the emission of the regulatory signal based on at least a sensed physiological signal or a change therein, or a command. In response to detection of a physiological parameter or a change in a physiological parameter, or a command, a signal is emitted from the device so as to increase expression of the open reading frame that is linked to the regulatable, field sensitive transcriptional control element in the expression cassette or an endogenous gene in cells of the mammal. In one embodiment, the regulatable, field sensitive transcriptional control element is regulated by heat or an electromagnetic field, or both. In one embodiment, the expression of the open reading frame in the expression cassette or endogenous gene is increased after the regulatory signal is emitted and optionally continues, at least for a period of time, after the regulatory signal ceases. In one embodiment, after the regulatory signal ends, the expression of the open reading frame decreases, e.g., to a lower level, or ceases, e.g., to pre-signal delivery levels.

Further provided is a method to control expression of an open reading frame present in cells of a mammal. The method includes delivering to a mammal a regulatory signal from a gene regulatory signal delivery device in an amount effective to regulate a regulatable, field sensitive transcriptional control element operably linked to an open reading frame for a gene product in cells of the mammal, thereby regulating the expression of the gene product. In one embodiment, a regulatory signal is delivered from the gene regulatory signal delivery device in response to a command, e.g., an external command or one from an implanted programmable device. In one embodiment, the mammal has or is at risk of a condition and delivery of the regulatory signal is in an amount effective to prevent, inhibit or treat the condition or at least one symptom thereof. For example, the condition is a cardiac condition and the expression of a selected open reading frame in a gene therapy vector in cells of a mammal having or at risk of the cardiac condition, may be upregulated by an implantable or external device, thereby altering one or more properties of the heart. In one embodiment, the condition is ischemia or remodeling, and the open reading frame in the expression cassette is selected so that expression of the open reading frame in cells of the mammal prevents, inhibits or treats ischemia or remodeling. In one embodiment, the method includes sensing in a mammal a physiological parameter or a change therein indicative of a predetermined cardiac condition, e.g., ischemia or remodeling, and delivering to the mammal a regulatory signal from the gene regulatory signal delivery device in response to at least the sensed physiological parameter or a change therein.

The invention thus provides methods of utilizing a device for maintaining control of expression of particular genes useful to prevent, inhibit or treat a condition, which genes are in genetically modified donor cells that are exogenously administered, unmodified endogenous cells or endogenous cells that are genetically modified, e.g., by recombinant virus administration. Thus, the systems and methods of the invention which employ sensors and/or diagnostic information allow for control of gene expression, thus providing for spatial, temporal and/or quantitative control of the gene product encoded by vector(s) or endogenous gene(s) in a mammal. While particular conditions and open reading frames for gene products useful to prevent, inhibit or treat that condition, e.g., gene products useful to treat severed nerves by inducing nerve regeneration, treat bone loss by inducing bone growth, or treat wounds by enhancing wound healing, or useful in diagnostics, are described herein, the invention is not limited to any condition or gene product.

### BRIEF DESCRIPTION OF THE DRAWINGS

The drawings illustrate generally, by way of example, but not by way of limitation, various embodiments discussed in the present document. The drawing are for illustrative purposes only and are not drawn to scale.

FIG. 1 is an illustration of an embodiment of a gene regulatory system and portions of an environment in which it is used.

FIG. 2 is a block diagram showing one embodiment of a circuit of portions of the gene regulatory system such as shown in FIG. 1.

FIG. 3 is a block diagram showing another embodiment of the circuit of portions of the gene regulatory system such as shown in FIG. 1.

FIG. 4 is an illustration of exemplary embodiments of an implantable gene regulatory signal delivery device of the gene regulatory system such as shown in FIG. 1.

FIG. 5 is an illustration of an embodiment of another gene regulatory system and portions of an environment in which it is used.

FIG. 6 is a block diagram showing one embodiment of a circuit of portions of the gene regulatory system such as shown in FIG. 5.

FIG. 7 is a schematic of the position of transcriptional control elements in the HSP70 promoter that are responsive to electromagnetic or thermal signals.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment.

General Overview

This document describes, among other things, methods and apparatus for the spatial, temporal and/or quantitative control of gene expression from an open reading frame in one or more exogenously administered genes (an expression cassette) or endogenous genes, expression that is useful to prevent, inhibit or treat, or detect, a particular condition, e.g., to detect hypoxia/ischemia. The gene includes at least one open reading frame encoding a desirable gene product operably linked to at least one regulatable, field sensitive transcriptional control element. A regulatable, field sensitive transcriptional control element includes a promoter, e.g., an inducible or repressible promoter, an enhancer, or a combination thereof. In one embodiment, the gene product encoded by the open reading frame is a protein or glycoprotein which is secreted or otherwise released from a cell, or a transcription factor which binds to a promoter linked to an open reading frame for a gene product, gene products including, but not limited to, a growth factor, e.g., vascular endothelial growth factor (VEGF), bone morphogenetic protein (BMP), or nerve growth factor (NGF), a survival or cardioprotective factor, such as a member of the bcl-2 family, Akt or a homolog or ortholog thereof, or another gene product with beneficial properties. For exogenously administered genes, the open reading frame may be present in a recombinant virus, isolated nucleic acid or donor cells. In one embodiment, donor cells, e.g., autologous cells, are genetically modified ex vivo by introducing an expression cassette containing a regulatable, field sensitive transcriptional control element linked to a desirable open reading frame, to those cells. In yet another embodiment, endogenous cells are genetically modified in vivo, for instance, by employing recombinant virus. In one embodiment, the expression of the exogenous or endogenous gene in vivo prevents, inhibits or treats at least one symptom of a particular condition. In one embodiment, the condition is a cardiac condition and the expression of the exogenous or endogenous gene(s) in a mammal having or at risk of the cardiac condition, which is controlled by an implantable or external device, beneficially alters at least one symptom of the condition.

The devices and apparatus of the invention which emit thermal or electromagnetic signals are useful in a variety of therapeutic treatments (wound healing, bone growth, nerve regeneration), as well as prevention of disease and protection from injury, e.g., induced by ischemia/reperfusion during cardiac surgery. In particular, the systems and methods of the invention may be used to prevent, inhibit or treat one or more symptoms of any condition amenable to treatment, prophylactic or otherwise, by gene expression. For instance, a system of the invention may be employed to enhance survival or suppress remodeling in a cardiac region impacted by MI or post MI conditioning in a cardiac region at the time of reperfusion.

Thus, regulated expression of particular genes can result in cardioprotection (e.g., resistance to ischemia-induced injury via endogenous HSP70 expression), enhanced biological pacemaker activity (e.g., non-invasive (remote) on/off switching of biological pacemaker expression), or diagnostic information based on time and target (location) specific activation of expression (e.g., pre-/post-conditioning immediately before or after metabolic stress). For instance, a device that emits a magnetic field, e.g., a field of less than 1 Gauss and 300 Hz, or heat can activate the expression of endogenous genes such as the HSP70 gene and other endogenous magnetic field responsive genes that function as protective agents by preventing adverse events, preserving a favorable environment, and/or repairing damage. In another embodiment, the activation of a gene-based biological pacemaker implanted in the heart may be driven by a magnetic field inducible promoter that can be controlled by an applied magnetic field. An example of a diagnostic system is the use of a biomarker to indicate hypoxia/ischemia. The system includes two expression cassettes. One expression cassette has a HSP70 promoter operably linked to a hypoxia inducible factor (HIF) open reading frame. Another cassette has a promoter with a hypoxia response element (HRE) linked to a reporter (e.g., secreted alkaline phosphatase). In the presence of an electromagnetic or thermal signal, HIF is expressed and activates expression of the reporter (e.g., SEAP) via the HRE promoter. Once the signal is removed, the biomarker is turned off.

Control of gene expression using a device of the invention that emits magnetic or thermal stimuli is both sensitive and rapidly responsive, and optionally non-invasive. For instance, an implantable device having a magnetic field generator provides local stimulation while an external device having a magnetic field generator, e.g., magnetic field generating clothing, beds, or hand held devices, provides regional stimulation in a non-invasive manner. Moreover, delivery of magnetic or thermal stimuli may be regulated by a device that is in communication with an implantable metabolic sensor, implantable ischemia detector, and/or an external controller.

This document discusses a gene expression regulatory system that includes a device which may be part of, or coupled to, a system which may include implantable medical devices. For example, the device may be part of or coupled to cardiovascular devices, e.g., catheters, leads and the like. In one embodiment, gene expression regulatory therapy is performed in conjunction with electrical therapy, such as pacing therapy. A specific example of an implantable medical device for use with a device of the invention is an implantable cardiac rhythm management (CRM) device. Several embodiments are presented below to provide examples of different apparatus and methods.

Definitions

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a sequence of interest for gene therapy. Vectors include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes. Vectors can also comprise other components or functionalities that further modulate gene delivery and/or gene expression, or that otherwise provide beneficial properties to the cells to which the vectors will be introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described.

“Gene delivery,” “gene transfer,” and the like as used herein, are terms referring to the introduction of an exogenous polynucleotide (sometimes referred to as a “transgene”), e.g., via a recombinant virus, into a host cell or by a genetically modified donor cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, iontophoresis, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the cell if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” or “genetically modified cell” is meant a cell containing a transgene. For example, a stem cell transformed with a vector containing an expression cassette can be used to produce a population of cells having altered phenotypic characteristics.

The term “wild-type” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

The term “transduction” denotes the delivery of a polynucleotide to a recipient cell either in vivo or in vitro, via a viral vector and preferably via a replication-defective viral vector, such as via a recombinant AAV a portion of which may subsequently be expressed in that cell.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation stimulations are located 3′ or downstream of the coding region.

A “gene,” “polynucleotide,” “coding region,” or “sequence” which “encodes” a particular gene product, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. Thus, a gene includes a polynucleotide which may include a full-length open reading frame which encodes a gene product (sense orientation) or a portion thereof (sense orientation) which encodes a gene product with substantially the same activity as the gene product encoded by the full-length open reading frame, the complement of the polynucleotide, e.g., the complement of the full-length open reading frame (antisense orientation) and optionally linked 5′ and/or 3′ noncoding sequence(s) or a portion thereof, e.g., an oligonucleotide, which is useful to inhibit transcription, stability or translation of a corresponding mRNA. A transcription termination sequence will usually be located 3′ to the gene sequence.

An “oligonucleotide” includes at least 7 nucleotides, preferably 15, and more preferably 20 or more sequential nucleotides, up to 100 nucleotides, either RNA or DNA, which correspond to the complement of the non-coding strand, or of the coding strand, of a selected mRNA, or which hybridize to the mRNA or DNA encoding the mRNA and remain stably bound under moderately stringent or highly stringent conditions, as defined by methods well known to the art, e.g., in Sambrook et al., A Laboratory Manual, Cold Spring Harbor Press (1989).

The term “control elements” refers collectively to promoter regions, polyadenylation stimulations, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence. Thus, a “promoter,” refers to a polynucleotide sequence that controls transcription of a gene or coding sequence to which it is operably linked. A large number of promoters, including constitutive, inducible and repressible promoters, from a variety of different sources, are well known in the art.

By “enhancer element” is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources are well known in the art. A number of polynucleotides which have promoter sequences (such as the commonly-used CMV promoter) also have enhancer sequences.

“Operably linked” refers to a juxtaposition, wherein the components so described are in a relationship permitting them to function in their intended manner. By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. Thus, a stimulation or targeting peptide sequence is operably linked to another protein if the resulting fusion is secreted from a cell as a result of the presence of a secretory stimulation peptide or into an organelle as a result of the presence of an organelle targeting peptide.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. An “animal” includes vertebrates such as mammals, avians, amphibians, reptiles and aquatic organisms including fish.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination stimulation, may also be included.

The term “exogenous,” when used in relation to a protein, gene or nucleic acid, e.g., polynucleotide, in a cell or organism refers to a protein, gene, or nucleic acid which has been introduced into the cell or organism by artificial or natural means, or in relation to a cell refers to a cell which was isolated and subsequently introduced to other cells or to an organism by artificial or natural means (a “donor” cell). An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide or virus refers to a nucleic acid sequence, peptide, polypeptide or virus that is identified and separated from at least one contaminant nucleic acid, polypeptide, virus or other biological component with which it is ordinarily associated in its natural source. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “peptide”, “polypeptide” and “protein” are used interchangeably herein unless otherwise distinguished to refer to polymers of amino acids of any length. These terms also include proteins that are post-translationally modified through reactions that include glycosylation, acetylation and phosphorylation.

By “growth factor” is meant an agent that, at least, promotes cell growth or induces phenotypic changes.

“Gene regulation” or “gene regulatory therapy” as used herein includes delivery of one or more gene regulatory stimulations to regulate gene expression in an expression cassette. The gene regulatory stimulations include stimulations that trigger a transcriptional control element, e.g., a promoter.

A “user” includes a physician or other caregiver using a gene regulatory system to treat a patient.

Expression Cassettes

The methods and apparatus of the invention include the use of nucleic acid vectors, recombinant virus, or genetically modified cells having an expression cassette to deliver genes to a mammal, the expression of which in vivo prevents, inhibits or treats at least one symptom of a particular condition, or is useful in diagnostics. The expression cassette includes at least one open reading frame encoding a gene product operably linked to at least one regulatable, field sensitive transcriptional control element. The methods and apparatus also employ a gene regulatory signal delivery device that emits a signal including, but not limited to, thermal or electromagnetic energy, which signal is recognized by the regulatable, field sensitive transcriptional control element. A regulatable, field sensitive transcriptional control element includes a promoter, e.g., an inducible or repressible promoter, an enhancer, or a combination thereof. For instance, the promoter may be responsive to certain temperatures. Examples of regulatable transcriptional control elements are discussed in U.S. patent application Ser. No. 10/788,906, entitled “METHOD AND APPARATUS FOR DEVICE CONTROLLED GENE EXPRESSION,” filed on Feb. 27, 2005, assigned to Cardiac Pacemakers, Inc., which is incorporated herein by reference in its entirety. Examples of elements useful in the methods of the invention include, but are not limited to, those sensitive to electromagnetic fields, e.g., those present in metallothionein I or II, c-myc, c-fos, jun, and HSP70 promoters (Lin et al., J. Cell. Biochem., 81:143 (2001); Lin et al., J. Cell. Biochem., 54:281 (1994); Goodman et al., J. Cell Physiol., 192:16 (2002); U.S. published application 20020099026)), or heat, e.g., heat shock promoters.

In some embodiments, cell- or tissue-specific control elements, such as muscle-specific and inducible promoters, enhancers and the like, will be of particular use, e.g., in conjunction with regulatable, field sensitive transcriptional control elements. Such control elements include, but are not limited to, those derived from the actin and myosin gene families, such as from the myoD gene family (Weintraub et al., Science, 251, 761 (1991)); the myocyte-specific enhancer binding factor MEF-2 (Cserjesi and Olson, Mol. Cell Biol., 11, 4854 (1991)); control elements derived from the human skeletal actin gene (Muscat et al., Mol. Cell Bio., 7, 4089 (1987)) and the cardiac actin gene; muscle creatine kinase sequence elements (Johnson et al., Mol. Cell Biol., 9, 3393 (1989)) and the murine creatine kinase enhancer (mCK) element; control elements derived from the skeletal fast-twitch troponin C gene, the slow-twitch cardiac troponin C gene and the slow-twitch troponin I genes. Cardiac cell restricted promoters include but are not limited to promoters from the following genes: a α-myosin heavy chain gene, e.g., a ventricular α-myosin heavy chain gene, β-myosin heavy chain gene, e.g., a ventricular β-myosin heavy chain gene, myosin light chain 2v gene, e.g., a ventricular myosin light chain 2 gene, myosin light chain 2a gene, e.g., a ventricular myosin light chain 2 gene, cardiomyocyte-restricted cardiac ankyrin repeat protein (CARP) gene, cardiac α-actin gene, cardiac m2 muscarinic acetylcholine gene, ANP gene, BNP gene, cardiac troponin C gene, cardiac troponin I gene, cardiac troponin T gene, cardiac sarcoplasmic reticulum Ca-ATPase gene, skeletal α-actin gene, as well as an artificial cardiac cell-specific promoter.

The response of the regulatable, field sensitive transcription control element to one or more intermittent stimulations, a prolonged stimulation or different levels of a stimulation, may be tested in a vector in vitro or in vivo. The vector may include the regulatable, field sensitive transcriptional control element linked to a marker gene, i.e., one which is readily detectable or capable of detection such as green fluorescent protein (GFP). For example, a vector having a transcriptional control element which is sensitive to heat, a HSP70 transcriptional control element, is linked to an open reading frame for a marker gene. The resulting expression cassette, e.g., one which is introduced to an adenovirus vector or to a plasmid vector, is employed to infect or transfect murine cells, e.g., murine cardiac cells, or heart sections, and heat applied. Then fluorescence in the cells or a lysate thereof is detected, and/or or vector specific RNA is measured, for instance, using RT-PCR, and optionally compared to data from control cells. Similarly, a vector having a promoter which is sensitive to an electromagnetic field is linked to an open reading frame, and the phenotype of cells transduced with the vector compared to control cells. Vectors may also be introduced to a non-human large animal model, e.g., pigs, to determine the level and spatial expression of the exogenously introduced gene in response to stimulations, e.g., an electromagnetic field, from an implantable or external device.

Open reading frames useful in vectors in vivo include those for growth factors, Akt, potassium channel proteins such as HCN, KCNA5(Kv1.5), KCND2(Kv4.2), KCND3 (Kv 4.3, Iₜₒ), KCNE1 (minK), KCNE2, KCNQ1, as well as K+ inwardly rectifying channel such as Kir3.1 (KCNJ3), KCNH2 (HERG, Iₖᵣ), Kir3.4, Kir6.1 and Kir6.2. In one embodiment, the open reading frame encodes acidic fibroblast growth factor (aFGF), basic fibroblast growth factor (bFGF), fibroblast growth factor 3 (FGF-3), fibroblast growth factor 4 (FGF-4), fibroblast growth factor 5 (FGF-5), fibroblast growth factor 6 (FGF-6), fibroblast growth factor 7 (FGF-7), fibroblast growth factor 8 (FGF-8), fibroblast growth factor 9 (FGF-9), angiogenin 1, angiogenin 2, platelet-derived endothelial-cell growth factor (PD-ECGF), transforming growth factor-α (TGF-α), transforming growth factor-β (TGF-β), e.g., TGF-beta₁, tumor necrosis factor-α (TNF-α), vascular endothelial growth factor B (VEGF-B), vascular endothelial growth factor C (VEGF-C), vascular endothelial growth factor D (VEGF-D), vascular endothelial growth factor E (VEGF-E), vascular endothelial growth factor F (VEGF-F), platelet derived growth factor (PDGF), e.g., PDGF-AA, PDGF-AB or PDGF-BB, vascular endothelial growth factor (VEGF), e.g., VEGF₁₄₅, VEGF₁₂₁, VEGF₁₂₀, VEGF₁₆₄, VEGF₁₆₅, VEGF₁₈₉, and VEGF₂₀₆, IGF-1, platelet-derived growth factor (PDGF), epidermal growth factor (EGF), macrophage colony-stimulating factor, endothelial growth factor, heat shock related proteins, e.g., HSP such as HSP70i, HSP27, HSP40 or HSP60, bone morphogenetic protein, e.g., BMP-2, BMP-4, BMP-17, or BMP-18, hypoxia inducible factor (HIF) 1α, and the like.

For instance, open reading frames for expression of antioxidants, e.g., HO-1, SOD, catalase or GPx, heat shock protein, e.g., HSP70, HSP90 or HSP27, survival genes, e.g., Bcl2, Akt or HGF, or for gene products associated with coronary vessel tone, e.g., eNOS, or adenosine P1 or P3 receptors, or to inhibit expression of inflammatory cytokines, adhesion molecules or transcription factors, such as ICAM, VCAM, TNF-α or NF-kB, or pro-apoptotic genes, e.g., Bad or caspase inhibitorp53, may be useful for MI. For coronary artery disease, expression of VEGF₁₂₁, VEGF₁₆₅, FGF-1, FGF-2, FGF-4, FGF-5, HGF, Ang-1, MCP-1, G-CSF, PDGF-BB, IGF-1, IGF-2, HIF-1α/VP16, egr-1 or Prox-1, may be useful to inhibit or treat coronary artery disease.

For heart failure, expression of beta-adrenoceptor or SERCA2A, or inhibition of expression of BARK or phospholamban, may be therapeutic. For inherited heart diseases such as arrhythmias or channelopathies, or cardiomyoplasty, those disorders may be inhibited or treated by altering expression of SCN5A, I_{K}, HERG, KCNE1, Gαi₂ or Kir2, or by expressing sarcomeric proteins or sarcoglycans, respectively. Congenital heart disease may be inhibited or treated by expression of endoglin, NKx2.5, TBX5 or TFAP2B.

To inhibit or treat vascular diseases associated with altered vascular tone, expression of kallikrein, ENOS, ANP, CNP, HO-1 or ecSOD (for vasodilation) or inhibition of expression of ACE, AGT or AT₁ (for vasoconstration), may be employed. To stabilize plaque, CD40 may be expressed. To promote cholesterol homeostasis, LDL-R, lipoprotein lipase, hepatic lipase, Apo-E, VLDL-R, SR-B1 or Apo-A1 may be expressed. Inhibition of the expression of the following may promote thromboprotection: PAI-1, plasminogen activator, tissue factor, MCP-1, t-PA, hirudin, urokinase, tissue factor pathway inhibitor, thrombomodulin, COX-1, PGI₂ synthase, eNOS, iNOS, HO-1 or SOD.

To alter vascular cell proliferation, cell cycle proteins, e.g., p16, p27, p21, p53, Rb, Cdc2, cdk2, c-myb, c-myc, PCNA or E2F, or gene products such as antiproliferative gene products, thymidine kinase, eNOS, iNOS, ecSOD or HO-1, may be expressed or the expression of transcription factors (TF), cytokines, apoptotic or signaling molecules, such as NF-kB, BcL-X_{L}, Fas ligand, Gax, GATA-6, β-interferon or VEGF, may be inhibited. Also, the expression of cytoproliferative genes, such as HO-1 or SOD, may protect the vasculature.

In one embodiment, to alter nerve function, isolated nucleic acid, recombinant virus or genetically altered cells may contain an expression cassette of the invention encoding galectin-1, proteins involved in steroidogenesis for injured nerves, e.g., StAB, PBrL, or aromatose, GAD-43, krox 24/EGR 1, T-alpha-1 tubulin, L1, nerve growth factor (NGF), galanin, sempaphorin 3A, brain derived neutrotrophic factor, neurotrophin-3, neurotrophin 4/5, insulin growth factor, ciliary neurotrophic factor, glial cell derived neurotrophic factor (GDNF), and the like.

In another embodiment, to alter bone regeneration, isolated nucleic acid, recombinant virus or genetically altered cells may contain an expression cassette of the invention encoding insulin-like growth factor I and II, osteoclastopoietic factor, TGF-beta, bone morphogenetic proteins (BMPs), e.g., BMP 3, 4, 7 or 8, PDGF, connective tissue growth factor, stanniocalcin, parathyroid hormone related protein, adrenomedullin, Ets-1 or -2, MIP-1 alpha, ANP, BNP, and the like

In yet another embodiment, to alter wound healing, isolated nucleic acid, recombinant virus or genetically altered cells may contain an expression cassette of the invention encoding matrix metalloproteases (MMPs), activin, a chemokine, a cytokines, cell migration inducing proteins, Akt, keratinocyte growth factor, FGF-7, connective tissue growth factor, PDGF, TGF-beta, platelet activating factor, COX-2, and the like.

Gene Delivery Vehicles

The exogenous gene may be present in a recombinant virus, isolated nucleic acid (e.g., a plasmid), or donor cells. Sources for donor cells include but are not limited to bone marrow-derived cells, e.g., mesenchymal cells and stromal cells, smooth muscle cells, fibroblasts, keratinocytes, SP cells, pluripotent cells or totipotent cells, e.g., teratoma cells, hematopoietic stem cells, for instance, cells from cord blood and isolated CD34⁺ cells, multipotent adult progenitor cells, adult stem cells, embryonic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. In one embodiment, the donor cells are autologous cells, however, non-autologous cells, e.g., xenogeneic cells, may be employed. The donor cells can be expanded in vitro to provide an expanded population of donor cells for administration to a host mammal. Sources of donor cells and methods of culturing those cells are known to the art.

Donor cells may be treated in vitro by subjecting them to mechanical, electrical, or biological conditioning, or any combination thereof, as described in U.S. patent application Ser. No. 10/722,115, entitled “METHOD AND APPARATUS FOR CELL AND ELECTRICAL THERAPY OF LIVING TISSUE”, which is incorporated by reference herein, conditioning which may include continuous or intermittent exposure to the exogenous stimuli. For instance, biological conditioning includes subjecting donor cells to exogenous agents, e.g., differentiation factors, growth factors, angiogenic proteins, survival factors, and cytokines, as well as to expression cassettes including encoding a gene product including, but not limited to, a growth factor, a survival factor, and the like. In one embodiment, donor cells, e.g., autologous or nonautologous cells, such as autologous myocardial cells, are genetically modified for use in the system of the invention ex vivo by introducing an expression cassette containing a regulatable, field sensitive transcriptional control element linked to a desirable open reading frame, for example, an open reading frame for a prosurvival factor, to those cells. In another embodiment, donor cells are genetically modified in vivo, for instance, by employing recombinant virus.

Delivery of an expression cassette to donor cells may be accomplished by any means, e.g., transfection with naked or plasmid DNA, liposomes, calcium-mediated transformation, electroporation, or transduction, e.g., using recombinant viruses, for instance, via adenovirus, adeno-associated virus, retrovirus or lentivirus vectors. A number of transfection techniques are generally known in the art. See, e.g., Graham et al., Virology, 52, 456 (1973), Sambrook et al., Molecular Cloning: A Laboratory Manual, Cold Spring Harbor Laboratories, New York (1989), Davis et al., Basic Methods in Molecular Biology, Elsevier (1986) and Chu et al., Gene, 13, 197 (1981). Particularly suitable transfection methods include calcium phosphate co-precipitation (Graham et al., Virol., 52, 456 (1973)), direct microinjection into cultured cells (Capecchi, Cell, 22, 479 (1980)), electroporation (Shigekawa et al., BioTechniques, 6, 742 (1988)), liposome-mediated gene transfer (Mannino et al., BioTechniques, 6, 682 (1988)), lipid-mediated transduction (Felgner et al., Proc. Natl. Acad. Sci. USA, 84 7413 (1987)), and nucleic acid delivery using high-velocity microprojectiles (Klein et al., Nature, 327, 70 (1987)). Preferred recombinant viruses to deliver exogenous genes to donor or host cells include recombinant lentiviruses, retroviruses, adenoviruses, adeno-associated viruses (AAV), and herpes viruses including cytomegalovirus.

Plasmid DNA is often referred to as “naked DNA” to indicate the absence of a more elaborate packaging system. Plasmid-based vectors are relatively nonimmunogenic and nonpathogenic, with the potential to stably integrate in the cellular genome, resulting in long-term gene expression in postmitotic cells in vivo. Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range.

Adenoviral vectors may be rendered replication-incompetent by deleting the early (E1A and E1B) genes responsible for viral gene expression from the genome and are stably maintained into the host cells in an extrachromosomal form. These viral vectors have the ability to transduce both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. In addition, adenoviral vectors can be produced at very high titers, allowing efficient gene transfer with small volumes of virus.

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to transduce replicating and nonreplicating cells and are believed to be nonpathogenic to humans.

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb). Herpesvirus vectors are particularly useful for delivery of large genes.

Viral and non-viral vectors may be present in liposomes or other lipid-containing complexes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes, and other macromolecular complexes capable of mediating delivery of a gene to a host cell. By way of illustration, liposomes and other lipid-containing delivery complexes can be used with the virus or isolated nucleic acid at the invention. The principles of the preparation and use of such complexes for gene delivery have been described in the art (see, e.g., Ledley, Human Gene Therapy, 6:1129 (1995); Miller et al., FASEB Journal, 9:190 (1995); Chonn et al., Curr. Opin. Biotech., 6:698 (1995); Schofield et al., British Med. Bull., 51:56 (1995); Brigham et al., J. Liposome Res., 3:31 (1993)).

Vectors can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Targeted vector constructs would thus include targeted delivery vectors and/or targeted vectors. Restricting delivery and/or expression can be beneficial as a means of further focusing the potential effects of gene therapy. The potential usefulness of further restricting delivery/expression depends in large part on the type of vector being used and the method and place of introduction of such vector. For instance, delivery of viral vectors via intracoronary injection to the myocardium has been observed to provide, in itself, highly targeted gene delivery. In addition, using vectors that do not result in transgene integration into a replicon of the host cell (such as adenovirus and numerous other vectors), is expected to exhibit relatively long transgene expression in cells that do not undergo rapid turnover. In contrast, expression in more rapidly dividing cells would tend to be decreased by cell division and turnover. However, other means of limiting delivery and/or expression can also be employed, in addition to or in place of the illustrated delivery method, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Miller, et al., FASEB Journal, 9:190 (1995); Chonn et al., Curr. Opin. Biotech., 6:698 (1995); Schofield et al., British Med. Bull., 51:56 (1995); Schreier, Pharmaceutica Acta Helvetiae, 68:145 (1994); Ledley, Human Gene Therapy, 6:1129 (1995); WO 95/34647; WO 95/28494; and WO 96/00295).

Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Expression Cassette Administration

Isolated DNA, recombinant virus or recombinant cells may be administered intravenously, transvenously, intramyocardially or by any other convenient route, and delivered by a needle, catheter, e.g., a catheter which includes an injection needle or infusion port, lead, or other suitable device. Generally any route of administration may be employed, including oral, mucosal, intramuscular, buccal and rectal administration. For certain vectors, certain routes of administration may be preferred. For instance, viruses, e.g., pseudotyped virus, and DNA- or virus-liposome, e.g., HVJ-liposome, may be administered by coronary infusion, while HVJ-liposome complexes may be delivered pericardially. Recombinant cells may also be delivered systemically, e.g., intravenously.

In one embodiment, administration may be by intracoronary injection to one or both coronary arteries (or to one or more saphenous vein or internal mammary artery grafts or other conduits) using an appropriate coronary catheter. A variety of catheters and delivery routes can be used to achieve intracoronary delivery, as is known in the art. For example, a variety of general purpose catheters, as well as modified catheters, suitable for use in the present invention are available from commercial suppliers. Also, where delivery to the myocardium is achieved by injection directly into a coronary artery, a number of approaches can be used to introduce a catheter into the coronary artery, as is known in the art. By way of illustration, a catheter can be conveniently introduced into a femoral artery and threaded retrograde through the iliac artery and abdominal aorta and into a coronary artery. Alternatively, a catheter can be first introduced into a brachial or carotid artery and threaded retrograde to a coronary artery. Detailed descriptions of these and other techniques can be found in the art (see, e.g., above, including: Topol, (ed.), The Textbook of Interventional Cardiology, 4th Ed. (Elsevier 2002); Rutherford, Vascular Surgery, 5th Ed. (W. B. Saunders Co. 2000); Wyngaarden et al. (eds.), The Cecil Textbook of Medicine, 22nd Ed. (W. B. Saunders, 2001); and Sabiston, The Textbook of Surgery, 16th Ed. (Elsevier 2000)).

Direct myocardial injection of plasmid DNA as well as virus vectors, e.g., adenoviral vectors, and cells including recombinant cells has been documented in a number of in vivo studies. This technique when employed with plasmid DNA or adenoviral vectors has been shown to result in effective transduction of cardiac myocytes. Thus, direct injection may be employed as an adjunct therapy in patients undergoing open-heart surgery or as a stand-alone procedure via a modified thorascope through a small incision. In one embodiment, this mode of administration is used to deliver a gene or gene product that would only require limited transfection efficiency to produce a significant therapeutic response, such as a gene that encodes for or leads to a secreted product (e.g., VEGF, endothelial nitric oxide synthase). In one embodiment, virus, e.g., pseudotyped, or DNA- or virus-liposome complexes may be delivered intramyocardially.

Intracoronary delivery of genetic material can result in transduction of approximately 30% of the myocytes predominantly in the distribution of the coronary artery. Parameters influencing the delivery of vectors via intracoronary perfusion and enhancing the proportion of myocardium transduced include a high coronary flow rate, longer exposure time, vector concentration, and temperature. Gene delivery to a substantially greater percent of the myocardium may be enhanced by administering the gene in a low-calcium, high-serotonin mixture (Donahue et al., Nat. Med., 6:1395 (2000)). The potential use of this approach for gene therapy for heart failure may be increased by the use of specific proteins that enhance myocardial uptake of vectors (e.g., cardiac troponin T).

Improved methods of catheter-based gene delivery have been able to achieve almost complete transfection of the myocardium in vivo. Hajjar et al. (Proc. Natl. Acad. Sci. USA, 95:5251 (1998)) used a technique combining surgical catheter insertion through the left ventricular apex and across the aortic valve with perfusion of the gene of interest during cross-clamping of the aorta and pulmonary artery. This technique resulted in almost complete transduction of the heart and could serve as a protocol for the delivery of adjunctive gene therapy during open-heart surgery when the aorta can be cross-clamped.

Recombinant cells may also be delivered via catheter.

Gene delivery to the ventricular myocardium by injection of genetic material into the pericardium has shown efficient gene delivery to the epicardial layers of the myocardium. However, hyaluronidase and collagenase may enhance transduction without any detrimental effects on ventricular function. Recombinant cells may also be delivered pericardially.

Intravenous gene delivery may be efficacious for myocardial gene delivery. However, to improve targeted delivery and transduction efficiency of intravenously administered vectors, targeted vectors may be employed. In one embodiment, intravenous administration of DNA-liposome or antibody-DNA complexes may be employed.

Gene delivery can be performed by incorporating a gene delivery device or lumen into a lead such as a pacing lead, defibrillation lead, or pacing-defibrillation lead. An endocardial lead including a gene delivery device or lumen allows gene delivery to the endocardial layers of the myocardium. An epicardial lead including a gene delivery device or lumen allows gene delivery to the endocardial layers of the myocardium. A transvenous lead including a gene delivery device or lumen may also allow intravenous gene delivery.

Administration of an expression cassette of the invention in accordance with the present invention may be continuous or intermittent, depending, for example, upon the recipient's physiological condition, whether the purpose of the administration is therapeutic or prophylactic, and other factors known to skilled practitioners. The administration of the expression cassette may be essentially continuous over a preselected period of time or may be in a series of spaced doses. Both local and systemic administration is contemplated. The amount of expression cassette, and the amount of device based signal emitted to achieve a particular outcome will vary depending on various factors including, but not limited to, the open reading frame and control element chosen, the condition, patient specific parameters, e.g., height, weight and age, and whether prevention or treatment is to be achieved. The system of the invention is amenable to chronic use for prophylactic purposes

For viral vectors, at least about 10³ to 10⁷ viral particles, preferably about 10⁹ viral particles, and more preferably about 10¹¹ viral particles can be administered. The number of viral particles may, but preferably does not, exceed 10¹⁴. For delivery of donor cells, from about 10² to 10¹⁰, e.g., from 10³ to 10⁹, 10⁴ to 10⁸, or 10⁵ to 10⁷, cells may be administered. Agents which may enhance expression cassette, e.g., donor cells with the expression cassette, function or stimulate angiogenesis, such pyruvate, catecholamine stimulating agents, fibroblast growth factor, e.g., basic fibroblast growth factor, acidic fibroblast growth factor, fibroblast growth factor-4 and fibroblast growth factor-5, epidermal growth factor, platelet-derived growth factor, vascular endothelial growth factor (e.g., VEGF₁₂₁, VEGF₁₄₅, VEGF₁₆₅, VEGF₁₈₉ or VEGF₂₀₆), tissue growth factors and the like, may optionally be administered with the cassette, e.g., donor cells having the cassette, or administered separately. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

Vectors of the invention, e.g., isolated nucleic acid or recombinant virus having the expression cassette, may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors of the present invention may be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

One or more suitable unit dosage forms having the expression cassette of the invention, which may optionally be formulated for sustained release, can be administered. The formulations may, where appropriate, be conveniently presented in discrete unit dosage forms and may be prepared by any of the methods well known to pharmacy. Such methods may include the step of bringing into association the expression cassette with liquid carriers, solid matrices, semi-solid carriers, finely divided solid carriers or combinations thereof, and then, if necessary, introducing or shaping the product into the desired delivery system.

Pharmaceutical formulations containing the expression cassette can be prepared by procedures known in the art using well known and readily available ingredients. For example, the cassette can be formulated with common excipients, diluents, or carriers, and formed into tablets, capsules, suspensions, powders, and the like. The cassettes of the invention can also be formulated as elixirs or solutions for convenient oral administration or as solutions appropriate for parenteral administration, for instance by intramuscular, subcutaneous or intravenous routes.

The pharmaceutical formulations of the cassette can also take the form of an aqueous or anhydrous solution or dispersion, or alternatively the form of an emulsion or suspension.

Thus, the cassette, e.g., in a genetically modified donor cell or recombinant virus, may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the expression cassette is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the cassette may be administered via nose drops, a liquid spray, such as via a plastic bottle atomizer or metered-dose inhaler. Typical of atomizers are the Mistometer (Wintrop) and the Medihaler (Riker).

The local delivery of the cassette can also be by a variety of techniques which administer the cassette at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the cassettes may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The cassette may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

When desired, the above-described formulations can be adapted to give sustained release of the active ingredient employed, e.g., by combination with certain hydrophilic polymer matrices, e.g., comprising natural gels, synthetic polymer gels or mixtures thereof.

Additionally, the cassettes are well suited to formulation as sustained release dosage forms and the like. The coatings, envelopes, and protective matrices may be made, for example, from polymeric substances, such as polylactide-glycolates, liposomes, microemulsions, microparticles, nanoparticles, or waxes.

The formulations and compositions described herein may also contain other ingredients such as antimicrobial agents, or preservatives. Furthermore, as described herein the active ingredients may also be used in combination with other therapeutic agents or therapies.

### Exemplary Systems of the Invention

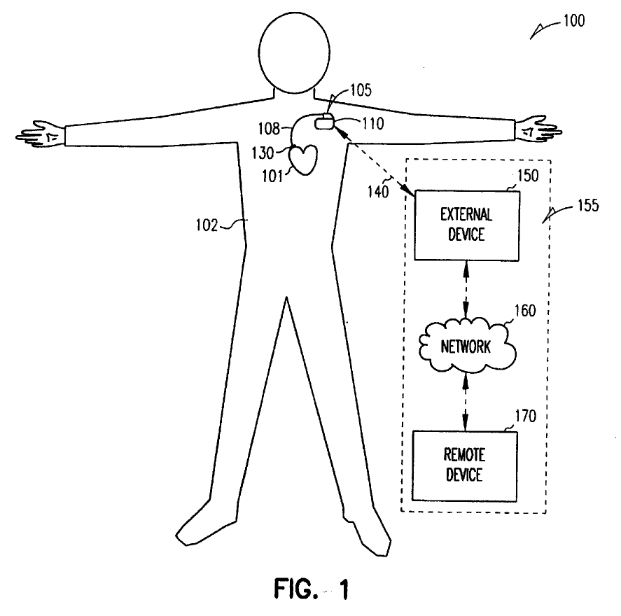

FIG. 1 is an illustration of an embodiment of a gene regulatory system 100 and portions of an environment in which system 100 is used. System 100 includes an implantable system 105, an external system 155, and a telemetry link 140 providing for communication between implantable system 105 and external system 155.

Implantable system 105 includes, among other things, an implantable CRM device 110, a lead system 108, and an implantable gene regulatory signal delivery device 130. As shown in FIG. 1, implantable CRM device 110 is implanted in a body 102. Implantable CRM device 110 is an implantable medical device that detects predetermined type cardiac events or therapy commands and may initiate one or more biologic therapies in response to the detection of each predetermined type cardiac event or therapy command. Implantable gene regulatory signal delivery device 130 delivers the biologic therapies by emitting one or more gene regulatory signals to a heart 101 or another one or more locations in body 102. The one or more gene regulatory signals includes one or both of electromagnetic (EM) and thermal stimuli to regulate a transcriptional control element, such as EM and/or heat responsive elements of the heat shock protein 70 (HSP70) promoter. Lead system 108 provides for access to one or more locations to which the one or more gene regulatory signals are delivered. In one embodiment, lead system 108 includes one or more leads providing for electrical connections between implantable CRM device 110 and implantable gene regulatory signal delivery device 130. In another embodiment, lead system 108 provides for the transmission of the one or more gene regulatory signals to the locations to which the signals are delivered. In various embodiments, implantable CRM device 110 is an implantable medical device that also includes a pacemaker, a cardioverter/defibrillator, a cardiac resynchronization therapy (CRT) device, a cardiac remodeling control therapy (RCT) device, a drug delivery device or a drug delivery controller, a cell therapy device, any combination of these devices, or any other implantable medical device. Lead system 108 further includes leads for sensing physiological signals and delivering pacing pulses, cardioversion/defibrillation shocks, and/or pharmaceutical or other substances.

External system 155 receives information acquired using implantable CRM device 110 and allows a user such as a physician or other caregiver to control the operation of implantable system 105. In one embodiment, external system 155 includes a programmer. In another embodiment, as illustrated in FIG. 1, external system 155 includes a patient monitoring system that includes an external device 150, a network 160, and a remote device 170. External device 150 is within the vicinity of implantable CRM device 110 and communicates with implantable CRM device 110 bi-directionally via telemetry link 140. Remote device 170 is in a remote location and communicates with external device 150 bi-directionally via network 160, thus allowing a user to monitor and treat a patient from a distant location.

System 100 allows the delivery of biologic therapies via emission of the one or more gene regulatory signals, to be triggered by any one of implantable CRM device 110, an implantable sensor or other component coupled to implantable CRM device 110, external device 150, and remote device 170. In one embodiment, implantable CRM device 110 triggers the delivery of a biologic therapy upon detecting a predetermined parameter or condition, such as the occurrence of an ischemic event or a particular level of metabolic activity. In another embodiment, external device 150 or remote device 170 triggers the delivery of the biologic therapy upon detecting an abnormal condition from a signal transmitted from implantable CRM device 110. In a specific embodiment, external system 155 includes a processor running a therapy decision algorithm to determine whether and when to trigger the delivery of the biologic therapy. In another specific embodiment, external system 155 includes a user interface to present signals acquired by implantable CRM device 155 and/or a detected abnormal condition or parameter to a user and receives commands from the user for triggering the delivery of the biologic therapy. In another specific embodiment, the user interface includes a user input incorporated into external device 150 to receive commands from the user and/or the patient treated with system 100. For example, the patient may be instructed to enter a command for the delivery of the biologic therapy when he senses certain symptoms, and another person near the patient may do the same upon observing the symptoms.

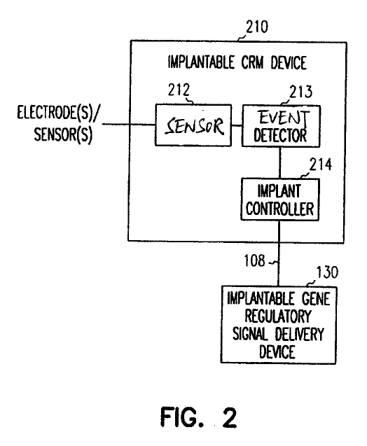

FIG. 2 is a block diagram showing one embodiment of the circuit of portions of system 100 including an implantable CRM device 210, lead system 108, and implantable gene regulatory signal delivery device 130. Implantable CRM device 210 represents a specific embodiment of implantable CRM device 110. In one embodiment, lead system 108 provides for an electrical connection between implantable CRM device 210 and implantable gene regulatory signal delivery device 130, such that implantable CRM device 210 transmits a voltage or current signal to control the delivery of a gene regulatory signal.

Implantable gene regulatory signal delivery device 130 receives a gene regulatory control signal from implantable CRM device 210 and, in response, delivers one or more gene regulatory signals in one or more forms of energy so as to regulate gene expression in an expression cassette or endogenous gene. To regulate the HSP70 promoter, the forms of energy include EM energy and thermal energy. In various embodiments, the one or more gene regulatory signals are each capable of regulating gene expression without inducing cardiac depolarization. For example, the one or more gene regulatory signals are each in a form of energy that is not known to excite myocardial tissue to cause depolarization of a cardiac chamber or in a level of intensity that does not excite myocardial tissue to cause depolarization of a cardiac chamber. In one embodiment, implantable gene regulatory signal delivery device 130 delivers one or more gene regulatory signals to the heart. In a specific embodiment, implantable gene regulatory signal delivery device 130 is an implantable device designed for placement in or on the heart, such as over an injured myocardial region.

Implantable CRM device 210 includes a sensor 212, an event detector 213, and an implant controller 214. Implantable CRM device 210 includes a hermetically sealed metal can to house at least a portion of the electronics of the device.

Sensor 212 senses the one or more physiological parameters, or changes in one or more of the parameters, indicative of one or more predetermined conditions through one or more electrodes and/or sensors. In one embodiment, the one or more predetermined conditions include one or more cardiac conditions. Examples of such cardiac condition include ischemia, reperfusion, and remodeling. In various embodiments, the electrode(s) include one or more electrodes incorporated into a lead of lead system 108, one or more electrodes incorporated onto implantable CRM device 210, and/or the metal can functioning as an electrode. In various embodiments, the sensor(s) includes one or more sensors incorporated into a lead of lead system 108, one or more sensors incorporated into implantable gene regulatory signal delivery device 130, one or more sensors incorporated onto implantable CRM device 210, one or more sensors housed within implantable CRM device 210, one or more sensors as separate implantable devices communicating with implantable CRM device 210, and/or one or more external (non-implantable) sensors communicating with implantable CRM device 210.

Event detector 213 detects the one or more predetermined conditions from the one or more physiological parameters or change(s) in one or more of the physiological parameters. Implant controller 214 is a microprocessor-based control circuit that controls the delivery of the one or more gene regulatory signals in response to the detection of at least one predetermined condition by event detector 213 and/or using the one or more parameters sensed by sensor 212. In various embodiments, event detector 213 and implant controller 214 are each implemented as portions of a microprocessor-based system.

In one embodiment, event detector 213 includes an ischemia detector and includes an ischemia analyzer running an automatic ischemia detection algorithm to detect the ischemic event from the one or more physiological parameters or changes therein. In one embodiment, event detector 213 produces an ischemia alert signal indicative of the detection of each ischemic event. In one embodiment, in response to the ischemia alert signal, implantable CRM device 210 produces an alarm signal, such as a predetermined audio tone, that is perceivable by the patient. In another embodiment, the ischemia signal is transmitted to external system 155 for producing an alarm signal and/or a warning message for the user and/or the patient.

In one embodiment, event detector 213 includes an ischemia detector that detects the ischemic events from one or more cardiac parameters or changes therein. Sensor 212 includes a cardiac sensing circuit. In a specific example, cardiac parameters are sensed using a wearable vest including embedded electrodes configured to sense surface biopotential parameters indicative of cardiac activities. The sensed surface biopotential parameters are transmitted to implantable CRM device 110 via telemetry. In another specific embodiment, event detector 213 detects the ischemic events from one or more wireless electrocardiograms (ECG). Sensor 212 includes a wireless ECG sensing circuit. A wireless ECG approximates the surface ECG and is acquired without using surface (skin contact) electrodes. An example of a circuit for sensing the wireless ECG is discussed in U.S. patent application Ser. No. 10/795,126, entitled “WIRELESS ECG IN IMPLANTABLE DEVICES,” filed on Mar. 5, 2004, assigned to Cardiac Pacemakers, Inc., which is incorporated by reference in its entirety. An example of a wireless ECG-based ischemia detector is discussed in U.S. patent application Ser. No. 11/079,744, entitled “CARDIAC ACTIVATION SEQUENCE MONITORING FOR ISCHEMIA DETECTION,” filed on Mar. 14, 2005, assigned to Cardiac Pacemakers, Inc., which is incorporated by reference in its entirety. In another embodiment, event detector 213 detects the ischemic events from one or more electrograms. Sensor 212 includes an electrogram sensing circuit. Examples of an electrogram-based ischemia detector are discussed in U.S. Pat. No. 6,108,577, entitled, “METHOD AND APPARATUS FOR DETECTING CHANGES IN ELECTROCARDIOGRAM SIGNALS,” and U.S. patent application Ser. No. 09/962,852, entitled “EVOKED RESPONSE SENSING FOR ISCHEMIA DETECTION,” filed on Sep. 25, 2001, both assigned to Cardiac Pacemakers, Inc., which are incorporated herein by reference in their entirety.

In another embodiment, event detector 213 includes an ischemia detector that detects the ischemic events from one or more impedance parameters. Sensor 212 includes an impedance sensing circuit to sense one or more impedance parameters each indicative of a cardiac impedance or a transthoracic impedance. Event detector 213 includes an electrical impedance based sensor using a low carrier frequency to detect the ischemic events from electrical impedance. Tissue electrical impedance has been shown to increase significantly during ischemia and decrease significantly after ischemia, as discussed in Dzwonczyk, et al. IEEE Trans. Biomed. Eng., 51(12): 2206-09 (2004). The ischemia detector senses low frequency electrical impedance between electrodes interposed in the heart, and detects the ischemia as abrupt changes in impedance (such as abrupt increases in value). In a specific embodiment, event detector 213 monitors complex impedance with concentration on the reactance to detect the ischemic events. Because ischemia induced changes in impedance occur predominantly in the reactive component, concentrating on the reactive component of the impedance provides for a high sensitivity of ischemia detection. In another specific embodiment, event detector 213 detects the ischemic events from multiple impedance parameters sensed through multiple electrodes positioned to monitor ventricular regional volumes or wall motion. The impedance parameters are indicative of changes in regional cardiac contractions resulting from ischemia. The ischemic events are detected by analyzing morphological and/or timing changes in the impedance parameters, such as by using a template matching technique.

In another embodiment, event detector 213 includes an ischemia detector that detects the ischemic events from one or more parameters indicative of heart sounds. Sensor 212 includes a heart sound sensing circuit. The heart sound sensing circuit senses the one or more parameters indicative of heart sounds using one or more sensors such as accelerometers and/or microphones. Such sensors are included in implantable CRM device 110 or incorporated into lead system 108. Event detector 213 detects the ischemic event by detecting predetermined type heart sounds, predetermined type heart sound components, predetermined type morphological characteristics of heart sounds, or other characteristics of heart sounds indicative of ischemia.

In another embodiment, event detector 213 includes an ischemia detector that detects the ischemic events from one or more pressure parameters. Sensor 212 includes a pressure sensing circuit coupled to one or more pressure sensors. In a specific embodiment, the pressure sensor is an implantable pressure sensor sensing a parameter indicative of an intracardiac or intravascular pressure whose characteristics are indicative of ischemia.

In another embodiment, event detector 213 includes an ischemia detector that detects the ischemic event from one or more acceleration parameters each indicative of regional cardiac wall motion. Sensor 212 includes a cardiac motion sensing circuit coupled to one or more accelerometers each incorporated into a portion of a lead positioned on or in the heart. The ischemia detector detects ischemia as an abrupt decrease in the amplitude of local cardiac accelerations.

In another embodiment, event detector 213 includes an ischemia detector that detects the ischemic event from a parameter indicative of heart rate variability (HRV). Sensor 212 includes an HRV sensing circuit to sense and produce a parameter which is representative of a HRV. HRV is the beat-to-beat variance in cardiac cycle length over a period of time. The HRV parameter includes any parameter being a measure of the HRV, including any qualitative expression of the beat-to-beat variance in cardiac cycle length over a period of time. In a specific embodiment, the HRV parameter includes the ratio of Low-Frequency (LF) HRV to High-Frequency (HF) HRV (LF/HF ratio). The LF HRV includes components of the HRV having frequencies between about 0.04 Hz and 0.15 Hz. The HF HRV includes components of the HRV having frequencies between about 0.15 Hz and 0.40 Hz. The ischemia detector detects ischemia when the LF/HF ratio exceeds a predetermined threshold. An example of an LF/HF ratio-based ischemia detector is discussed in U.S. patent application Ser. No. 10/669,168, entitled “METHOD FOR ISCHEMIA DETECTION BY IMPLANTABLE CARDIAC DEVICE,” filed on Sep. 23, 2003, assigned to Cardiac Pacemakers, Inc., which is incorporated by reference in its entirety.

In one embodiment, event detector 213 includes a metabolic level detector. Sensor 212 includes a metabolic sensor that senses a metabolic signal indicative of a cardiac metabolic level (rate of metabolism of cardiac cells). Examples of the metabolic sensor include a pH sensor, an oxygen pressure (PO₂) sensor, a carbon dioxide pressure (PCO₂) sensor, a glucose sensor, a creatine sensor, a C-creative protein sensor, a creatine kinase sensor, a creatine kinase-MB sensor, and any combination of such sensors. Event detector 213 determines the cardiac metabolic level from the metabolic signal and compares the cardiac metabolic level to one or more predetermined thresholds defining a normal cardiac metabolic range. The abnormal condition is detected when the cardiac metabolic level is outside of the normal cardiac metabolic range.

In one embodiment, event detector 213 includes an arrhythmia detector that detects an arrhythmia. Sensor 212 includes a cardiac sensing circuit that senses an electrogram, and event detector 213 detects an arrhythmia. In one embodiment, event detector 213 detects the arrhythmia by detecting heart rate and comparing the heart rate to one or more threshold rates. A bradycardia condition is detected when the heart rate falls below a bradycardia threshold. A tachycardia condition is detected when the heart rate exceeds a tachycardia threshold. In a further embodiment, event detector 213 detects the arrhythmia also by detecting morphological features of the electrogram to one or more predetermined templates. In a specific embodiment, event detector 213 includes an atrial fibrillation detector. In a specific embodiment, event detector 213 includes a ventricular fibrillation detector.

In one embodiment, event detector 213 includes an abnormal impedance detector that detects an abnormal impedance when an impedance is out of its normal range. For example, pulmonary edema, i.e., fluid retention in the lungs resulting from the decreased cardiac output, increases the pulmonary or thoracic impedance. Sensor 212 includes an impedance sensor to measure pulmonary impedance or impedance of a portion of the thoracic cavity. In a specific embodiment, event detector 213 produces the alert signal when the pulmonary or thoracic impedance exceeds a predetermined threshold impedance. In one embodiment, the impedance sensor is a respiratory sensor that senses the patient's minute ventilation. An example of an impedance sensor sensing minute ventilation is discussed in U.S. Pat. No. 6,459,929, “IMPLANTABLE CARDIAC RHYTHM MANAGEMENT DEVICE FOR ASSESSING STATUS OF CHF PATIENTS,” assigned to Cardiac Pacemakers, Inc., which is incorporated herein by reference in its entirety.

In one embodiment, event detector 213 includes an abnormal pressure detector that detects an abnormal pressure level when a blood pressure is outside of its normal range. Sensor 212 includes a pressure sensor. Arrhythmias and heart failure cause pressures in various portions of the cardiovascular system to deviate from their normal ranges. In a specific embodiment, event detector 213 includes a systolic dysfunction detector to detect an abnormal condition related to pressure during the systolic phase of a cardiac cycle. In another specific embodiment, event detector 213 includes a diastolic dysfunction detector to detect an abnormal condition related to pressure during the diastolic phase of a cardiac cycle. Examples of the pressure sensor include but are not limited to a left atrial (LA) pressure sensor, a left ventricular (LV) pressure sensor, an artery pressure sensor, and a pulmonary artery pressure sensor. Pulmonary edema results in elevated LA and pulmonary arterial pressures. A deteriorated LV results in decreased LV and arterial pressures. In various embodiments, event detector 213 detects an abnormal condition when the LA pressure exceeds a predetermined threshold LA pressure level, when the pulmonary arterial pressure exceeds a predetermined threshold pulmonary arterial pressure level, when the LV pressure falls below a predetermined threshold LV pressure level, and/or when the arterial pressure falls below a predetermined threshold LV pressure level. In other embodiments, event detector 213 derives a parameter from one of these pressures, such as a rate of change of a pressure, and produces a signal when the parameter deviates from its normal range. In one embodiment, the LV pressure sensor senses the LV pressure indirectly, by sensing a signal having known or predictable relationships with the LV pressure during all or a portion of the cardiac cycle. Examples of such a signal include but are not limited to an LA pressure and a coronary vein pressure. A specific example of measuring the LV pressure using a coronary vein pressure sensor is discussed in U.S. patent application Ser. No. 10/038,936, “METHOD AND APPARATUS FOR MEASURING LEFT VENTRICULAR PRESSURE,” filed on Jan. 4, 2002, assigned to Cardiac Pacemakers, Inc., which is hereby incorporated by reference in its entirety.

In one embodiment, event detector 213 includes a low stroke volume detector that detects a low stroke volume when the stroke volume falls below a predetermined threshold level. Sensor 212 includes a cardiac output or stroke volume sensor. Examples of stroke volume sensing are discussed in U.S. Pat. No. 4,686,987, “BIOMEDICAL METHOD AND APPARATUS FOR CONTROLLING THE ADMINISTRATION OF THERAPY TO A PATIENT IN RESPONSE TO CHANGES IN PHYSIOLOGIC DEMAND,” and U.S. Pat. No. 5,284,136, “DUAL INDIFFERENT ELECTRODE PACEMAKER,” both assigned to Cardiac Pacemakers, Inc., which are incorporated herein by reference in their entirety.

In one embodiment, event detector 213 includes an abnormal hormone level detector that detects an abnormal hormone level when a neurohormone level exceeds a predetermined threshold level. Sensor 212 includes a neural activity sensor to detect activities of the sympathetic nerve and/or the parasympathetic nerve. A significant decrease in cardiac output immediately stimulates sympathetic activities, as the autonomic nervous system attempts to compensate for deteriorated cardiac function. In a specific embodiment, the neural activity sensor includes a neurohormone sensor to sense a hormone level of the sympathetic nerve and/or the parasympathetic nerve. In another specific embodiment, the neural activity sensor includes an action potential recorder to sense the electrical activities in the sympathetic nerve and/or the parasympathetic nerve. Event detector 213 detects the abnormal hormone level on when the frequency of the electrical activities in the sympathetic nerve exceeds a predetermined threshold level. Examples of direct and indirect neural activity sensing are discussed in U.S. Pat. No. 5,042,497, “ARRHYTHMIA PREDICTION AND PREVENTION FOR IMPLANTED DEVICES,” assigned to Cardiac Pacemakers, Inc., which is hereby incorporated by reference in its entirety.

In one embodiment, event detector 213 includes an abnormal HRV detector that detects a low HRV when the HRV falls below a predetermined threshold level. Sensor 212 includes an HRV sensing circuit to sense and produce a parameter which is representative of HRV. Patients suffering acute decompensated heart failure exhibit abnormally low heart rate variability. An example of detecting the heart rate variability is discussed in U.S. Pat. No. 5,603,331, “DATA LOGGING SYSTEM FOR IMPLANTABLE CARDIAC DEVICE,” assigned to Cardiac Pacemakers, Inc., which is incorporated herein by reference in their entirety.

In one embodiment, event detector 213 includes an abnormal renal condition detector that detects an abnormal renal condition. Sensor 212 includes a renal function sensor. Acute decompensated heart failure results in peripheral edema primarily because of fluid retention of the kidneys that follows the reduction in cardiac output. The fluid retention is associated with reduced renal output, decreased glomerular filtration, and formation of angiotensin. Thus, in a specific embodiment, the renal function sensor includes a renal output sensor to sense a signal indicative of the renal output. Event detector 213 detects the abnormal renal condition when the sensed renal output falls below a predetermined threshold. In another specific embodiment, the renal function sensor includes a filtration rate sensor to sense a signal indicative of the glomerular filtration rate. Event detector 213 detects the abnormal renal condition when the sensed glomerular filtration rate falls below a predetermined threshold. In yet another specific embodiment, the renal function sensor includes a chemical sensor to sense a signal indicative of angiotensin II levels. Event detector 213 detects the abnormal renal condition when the sensed angiotensin II levels exceed a predetermined threshold level.

In one embodiment, event detector 213 includes an abnormal mechanical condition detector that detects an abnormal mechanical condition when the amplitude of a heart sounds or respiratory sound is out of its normal range. Sensor 212 includes an acoustic sensor being a heart sound sensor and/or a respiratory sound sensor. Arrhythmias and/or heart failure cause abnormal cardiac and pulmonary activity patterns and hence, deviation of heart sounds and respiratory sounds from their normal ranges of pattern and/or amplitude. For example, detection of the third heart sound (S3) is known to indicate heart failure. In a specific embodiment, event detector 213 detects the abnormal condition when the S3 amplitude or amount of S3 activity exceeds a predetermined threshold level. An example of using S3 activity to monitor for heart failure is discussed in U.S. patent application Ser. No. 10/746,874, “A THIRD HEART SOUND ACTIVITY INDEX FOR HEART FAILURE MONITORING,” filed on Dec. 24, 2003, assigned to Cardiac Pacemakers, Inc., which is hereby incorporated by reference in its entirety.

Embodiments of sensor 212 and event detector 213 are discussed in this document by way of example, but not by way of limitation. In various embodiment, sensor 212 and event detector 213 may include combinations of various sensors and detectors discussed above. Implant controller 214 controls the delivery of the one or more gene regulatory signals in response to the detection of any one or more of the conditions detected by event detector 213 as discussed above and/or using any one or more parameters sensed by sensor 212 as discussed above. Other methods and sensors for directly or indirectly detecting an abnormal condition treatable by the gene regulatory therapy are generally useable by gene regulatory system 100.

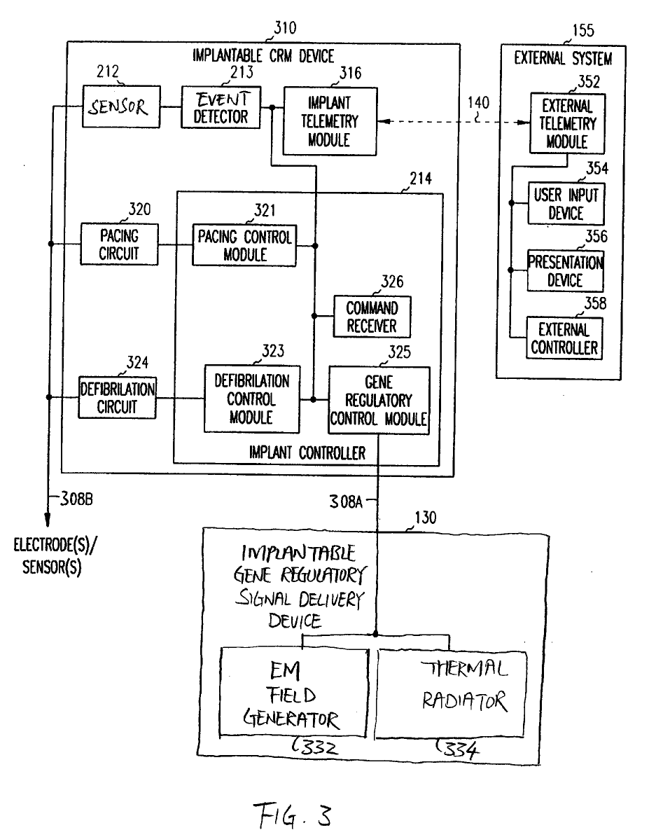

FIG. 3 is a block diagram showing another embodiment of the circuit of portions of system 100 including an implantable CRM device 310, lead system 108, implantable gene regulatory signal delivery device 130, and external system 155. Implantable CRM device 310 represents a specific embodiment of implantable CRM device 210 and includes pacing and defibrillation capabilities. In addition to controlling biologic therapies, implantable CRM device 310 delivers therapies including, but not limited to, one or more of bradyarrhythmia pacing, anti-tachyarrhythmia pacing, atrial and/or ventricular cardioversion/defibrillation, CRT, RCT, and drug delivery. However, such therapeutic capabilities are not necessary for system 100 to control gene therapy, and hence, are not necessary elements of implantable CRM device 310. In other words, implantable CRM device 310 can be an implantable pacemaker and/or defibrillator with additional functions including control of gene therapy, or it can be a dedicated implantable gene therapy controller.

In the embodiment illustrated in FIG. 3, implantable gene regulatory signal delivery device 130 includes an EM field generator 332 and a thermal radiator 334. In various embodiments, implantable gene regulatory signal delivery device 130 includes any one or both of EM field generator 332 and thermal radiator 334.

EM field generator 332 generates and emits an EM field that has frequency and strength parameters selected for regulating a field sensitive transcriptional control element, e.g., the HSP70 promoter. In one embodiment, EM field generator 332 includes an inductive coil. The intensity of the electromagnetic field is controlled by controlling the voltage across the coil and/or the current flowing through the inductive coil. In one embodiment, the frequency of the EM field is in a range of approximately 30 Hz to 300 Hz, with approximately 60 Hz being a specific example. The intensity of the EM filed is in a range of about 1 mGauss to 1000 mGauss with approximately 80 mGauss being a specific example.

Thermal radiator 334 emits a thermal energy that changes the tissue temperature to a point or range suitable for regulating a field sensitive transcriptional control element, e.g., the HSP70 promoter. In one embodiment, thermal radiator 334 includes a resistive element that is heated when an electrical current flows through it or as a voltage is applied across it. The tissue temperature is controlled by controlling the amplitude of the electrical current or voltage. In one embodiment, thermal radiator 334 is capable of rising the temperature of a portion of tissue by approximately 0° to 8° C.

In the embodiment illustrated in FIG. 3, implantable CRM device 310 includes sensor 212, event detector 213, implant controller 214, a pacing circuit 320, a defibrillation circuit 324, and an implant telemetry module 316. Pacing circuit 320 delivers pacing pulses to one or more cardiac regions as controlled by implant controller 214. Defibrillation circuit 324 delivers cardioversion/defibrillation shocks to one or more cardiac regions as controlled by implant controller 214. Implant controller 214 includes a gene regulation control module 325, a pacing control module 321, a defibrillation control module 323, and a command receiver 326. Gene regulation control module 325 generates the gene regulatory signal in response to a predetermined event detected by event detector 213 or in response to a gene regulatory command received by command receiver 326. Command receiver 326 receives the gene regulatory command from external system 155 via telemetry link 140. Pacing control module 321 controls the delivery of pacing pulses from pacing circuit 320 according to a bradyarrhythmia pacing algorithm, a CRT algorithm, and/or an RCT algorithm. Defibrillation control module 323 controls the delivery of cardioversion/defibrillation shocks from defibrillation circuit 324 when a tachyarrhythmic condition is detected. In one embodiment, defibrillation control module 323 includes an atrial defibrillation control module to control the delivery of cardioversion/defibrillation shocks to one or more of the atria. In one embodiment, defibrillation control module 323 includes a ventricular defibrillation control module to control the delivery of cardioversion/defibrillation shocks to one or more of the ventricles.

As illustrated in FIG. 3, a specific embodiment of lead system 108 includes one or more leads connecting implantable CRM device 310 and implantable gene regulatory signal delivery device 130, referenced as lead system 308A, and pacing leads, defibrillation leads, pacing-defibrillation leads, or any combination of such leads, referenced as lead system 308B. Lead system 308B allows sensing of electrical parameters from various regions of heart 101 and/or delivery of pacing pulses and/or defibrillation shocks to various regions of heart 101. The various regions of heart 101 includes regions within or about the right atrium (RA), left atrium (LA), right ventricle (RV), and left ventricle (LV). In a specific embodiment, lead system 308B includes one or more transvenous leads each having at least one sensing-pacing or defibrillation electrode disposed within heart 101. In a specific embodiment, lead system 308B includes one or more epicardial leads each having at least one sensing-pacing or defibrillation electrode disposed on heart 101. In a specific embodiment, lead system 308B includes at least one atrial defibrillation electrode disposed in or about one or both of the atria to allow atrial defibrillation. In a specific embodiment, lead system 308B includes at least one ventricular defibrillation electrode disposed in or about one or both of the ventricles to allow ventricular defibrillation. In an alternative embodiment, implantable CRM device 310 and implantable gene regulatory signal delivery device 130 communicate via a telemetry link, eliminating the need of electrically connecting the two implantable devices.

External system 155 includes an external telemetry module 352, an external user input device 354, a presentation device 356, and an external controller 358. In the embodiment in which external system 155 includes a patient management system, these system components distribute in one or more of external device 150, network 160, and remote device 170, depending on design and medical considerations. User input device 354 receives commands from the user and/or the patient to control the delivery of the biologic therapy, i.e., the delivery of the one or more gene regulatory signals. Presentation device 356 displays or otherwise presents parameters acquired and/or information regarding events detected by implantable CRM device 310. External controller 358 controls the operation of external system 155. In one embodiment, external controller 358 includes an ischemia alert signal receiver that receives the ischemia alert signal produced by event detector 213 and transmitted via telemetry link 140. Presentation device 356 presents an alarm signal and/or a warning message in response to the ischemia alert signal. In a specific embodiment, presentation device 356 includes a speaker to produce an audible alarm signal and/or an audible warning message in response to the ischemia alert signal. The audible alarm signal and/or warning message call for immediate attention of the patient or a physician or other caregiver. In a further specific embodiment, presentation device 356 also includes a display to visually display the alarm signal and/or warning message. In one embodiment, external controller 358 further provides automatic control of operations of implantable CRM device 310. In one embodiment, user input device 352 receives the gene regulatory command entered by the user based on observations of the parameters and/or abnormal conditions presented by presentation device 356. In another embodiment, user input device 352 receives the gene regulatory command entered by the patient when the patient physically senses a symptom indicative of an immediate need for the gene regulatory therapy, or entered by a person near the patient who observes a symptom indicative of the immediate need for the biologic therapy. In a further embodiment, external controller 358 automatically analyzes the signals acquired and/or events detected by implantable CRM device 310 and generate the gene regulatory command when deemed necessary based on the result of the analysis.

Telemetry link 140 is a wireless bidirectional data transmission link supported by implant telemetry module 316 and external telemetry module 352. In one embodiment, telemetry link 140 is an inductive couple formed when two coils—one connected to implant telemetry module 316 and the other connected to external telemetry module 352—are placed near each other. In another embodiment, telemetry link 140 is a far-field radio-frequency telemetry link allowing implantable CRM device 310 and external system 155 to communicate over a telemetry range that is at least ten feet.

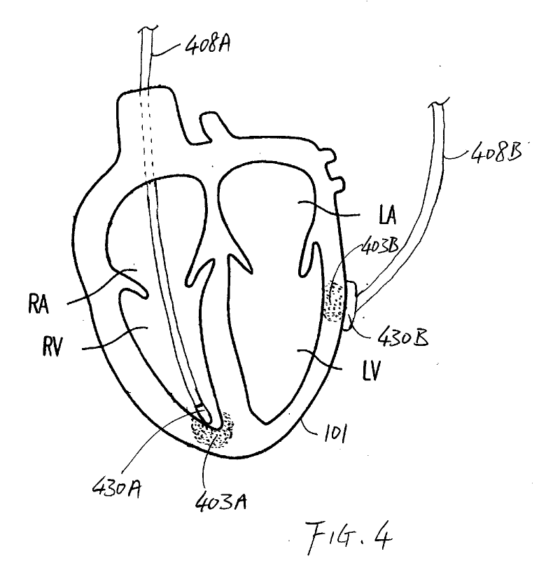

FIG. 4 is an illustration of exemplary embodiments of implantable gene regulatory signal delivery device 130 shown as implantable gene regulatory signal delivery device 430A and implantable gene regulatory signal delivery device 430B. Implantable gene regulatory signal delivery device 430A is incorporated into a distal end of an endocardial lead 408A for placement within a heart chamber (such as RV, as illustrated) over an injured myocardial region 403A of heart 101. Implantable gene regulatory signal delivery device 430B is incorporated into a distal end of an epicardial lead 408B such that it can be placed on heart 101 over an injured myocardial region 403B of heart 101. Leads 408A and 408B are each a specific embodiment of a lead of lead system 108. In one embodiment, in addition to providing for control of implantable gene regulatory signal delivery devices 430A and 430B, leads 408A and 408B each allow for one or more of sensing physiological signals, delivering pacing pulses, delivering cardioversion/defibrillation shocks, and delivering pharmaceutical or other substances.

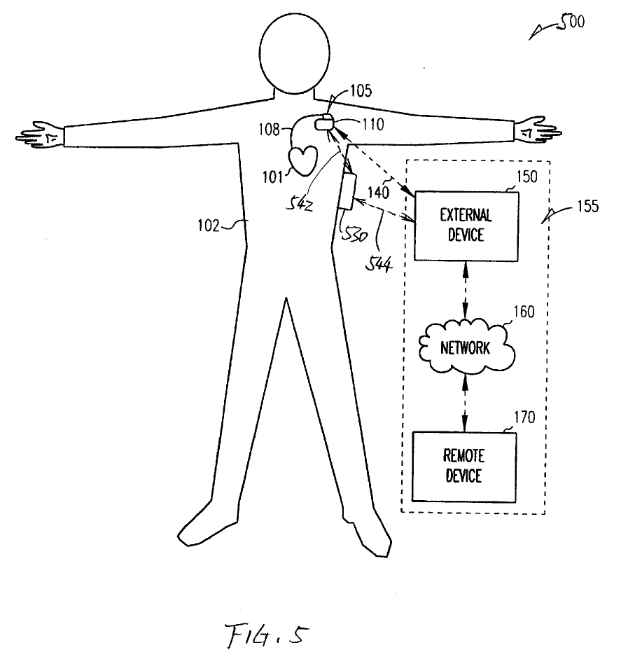

FIG. 5 is an illustration of an embodiment of a gene regulatory system 500 and portions of an environment in which system 500 is used. System 500 includes implantable system 105, external system 155, telemetry link 140, and an external gene regulatory signal delivery device 530. In the embodiment as illustrated in FIG. 5, system 500 also includes a telemetry link 542 providing for communication between implantable system 105 and external gene regulatory signal delivery device 530 and a telemetry link 544 providing for communication between external system 155 and external gene regulatory signal delivery device 530. In other embodiments, system 500 includes either one of, or none of, telemetry links 542 and 544. In one embodiment, an electrical connection, such as a cable, provides for the communication between external system 155 and external gene regulatory signal delivery device 530.

As illustrated in FIG. 5, system 500 is similar to system 100 except that external gene regulatory signal delivery device 530 delivers the biologic therapies by emitting the one or more gene regulatory signals, instead of implantable gene regulatory signal delivery device 130. In various embodiments, external gene regulatory signal delivery device 530 may be constructed as a patch for attached to skin, a handheld device, a device embedded in a bed or chair, or a device incorporated or attached to clothing. In one embodiment, external gene regulatory signal delivery device 530 is to be placed or held near heart 101 for delivering the biologic therapies, such as by placing on skin of body 102 over heart 101.

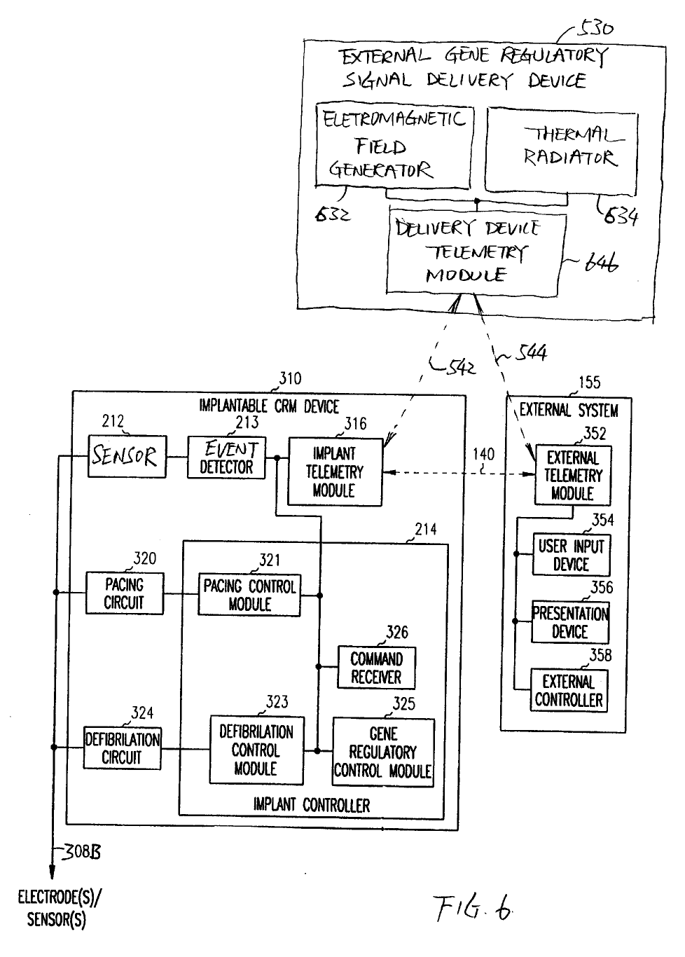

FIG. 6 is a block diagram showing an embodiment of the circuit of portions of system 500 including an implantable CRM device 310, lead system 308B, external system 155, and external gene regulatory signal delivery device 530. The circuit of FIG. 6 is similar to the circuit of FIG. 3 except for external gene regulatory signal delivery device 530, which replaces implantable gene regulatory signal delivery device 130.

In the embodiment illustrated in FIG. 6, external gene regulatory signal delivery device 530 includes an EM field generator 632, a thermal radiator 634, and a delivery device telemetry module 646. In various embodiments, external gene regulatory signal delivery device 530 includes any one or both of EM field generator 332 and thermal radiator 334, and optionally delivery device telemetry module 646.

EM field generator 632 generates and emits an EM field that has frequency and strength parameters selected for regulating a EM responsive transcriptional control element. In one embodiment, EM field generator 632 includes an inductive coil. The intensity of the electromagnetic field is controlled by controlling the voltage across the coil and/or the current flowing through the inductive coil. In one embodiment, the frequency of the EM field is in a range of approximately 30 Hz to 300 Hz, with approximately 60 Hz being a specific example. The intensity of the EM field is in a range of about 1 mGauss to 1000 mGauss with approximately 80 mGauss being a specific example. In a specific embodiment, the intensity is adjusted for transmitting a required or desirable amount of EM energy to a portion of heart 101 through skin and other tissue.

Thermal radiator 634 emits a thermal energy that changes the tissue temperature to a point or range suitable for regulating a temperature sensitive transcriptional control element. In one embodiment, thermal radiator 634 includes a resistive element that is heated when an electrical current flows through it or as a voltage is applied across it. The tissue temperature is controlled by controlling the amplitude of the electrical current or voltage. In one embodiment, thermal radiator 634 is capable of rising the temperature of a portion of tissue by approximately 0° to 8° C. In a specific embodiment, thermal radiator 634 transmits thermal energy through skin and other tissue to heat a portion of heart 101 to a required or desirable temperature.

In the embodiment illustrated in FIG. 6, delivering device telemetry module 646 supports telemetry links 542 and 544. This allows external gene regulatory signal delivery device 530 to deliver the one or more gene regulatory signals in response to a gene regulatory signal generated by gene regulation control module 325 or a gene regulatory command receive from external system 155. In another embodiment, system 500 does not include telemetry link 544. Gene regulation control module 325 generates the gene regulatory signal in response to a predetermined event detected by event detector 213 or in response to a gene regulatory command received by command receiver 326. The gene regulatory signal is then transmitted to external gene regulatory signal delivery device 530 via telemetry link 542 to cause the delivery of the one or more gene regulatory signals. In another embodiment, system 500 does not include telemetry link 542. External system 155 issues a gene regulatory command in response to a command entered by a user, such as a physician or other caregiver or the patient, or a reception of a signal indicative of a detection of the predetermined condition by event detector 213. The gene regulatory command is then transmitted to external gene regulatory signal delivery device 530 via telemetry link 544 to cause the delivery of the one or more gene regulatory signals. In another embodiment, external gene regulatory signal delivery device 530 receives the gene regulatory command from external system 155 through an electrical connection such as a cable. In another embodiment, external gene regulatory signal delivery device 530 is directly controlled by the user, eliminating the necessity for telemetry link 542 or 544.

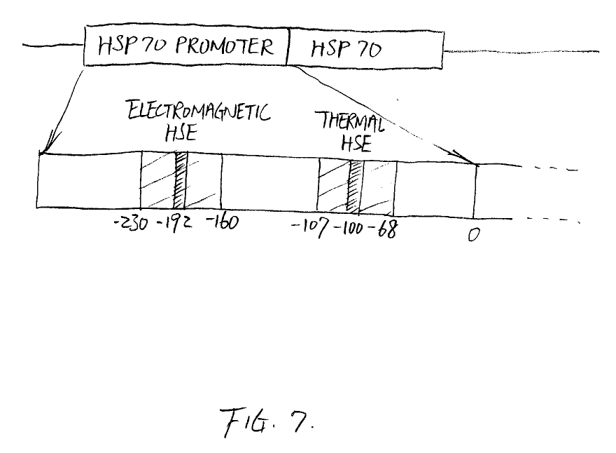

In one embodiment, a system of the invention includes devices that provide EM or heat stimulus and employs genetic materials that contain EM and/or heat responsive elements of the HSP70 transcriptional control region (FIG. 7). HSP70 is expressed in response to various stress stimuli, including heat and EM signals, and the HSP70 promoter in an expression cassette can be designed so that the promoter responds to either of the signals or both at one time. A key responsive sequence (-nCTCTn-) has been identified as the EM responsive element (EMRE) in the HSP70 promoter. Repetition of this EMRE in a genetically engineered construct allows amplification of EM responsiveness, and thus provides a way to quantitatively regulate the therapeutic effect and EM field strength.

In one embodiment, expression of an open reading frame linked to a HSP70 promoter in an expression cassette that is delivered to a target area in a mammal, can be controlled via either EM or thermal stimuli. For instance, expression of Akt linked to a HSP70 promoter in an expression cassette that is delivered to a region of the heart impacted by ischemia/myocardial infarction, can be controlled by either magnetic or heat stimuli, resulting in enhanced survival of the myocardium at risk. For instance, an implantable device in the mammal detects ischemia and then emits heat or EM energy, which induces expression from the promoter so as to yield an effective amount of Akt, e.g., a cardioprotective amount.

In another embodiment, a mammal has a cardiac condition characterized by aberrant pacemaker activity. The mammal is contacted with a gene therapy vector or genetically modified donor cells having an open reading frame encoding a hyperpolarization activated pacemaker channel protein (HCN) operably linked to an EM field sensitive promoter. In one embodiment, the vector or genetically modified donor cells are administered to one or more regions of the heart. An implantable device with a sensor detects decreased pacemaker activity in the mammal, and emits EM energy of a frequency and strength which induces expression from the promoter linked to the HCN open reading frame. The upregulated HCN expression in cardiac tissue yields cells with enhanced pacemaker activity.

In yet another embodiment, wound healing, bone growth or nerve regeneration may be enhanced in a mammal having a system of the invention. For example, to enhance wound healing, donor cells such as keratinocytes or fibroblasts, e.g., autologous keratinocytes or fibroblasts cells, are genetically modified ex vivo to express a growth factor. The genetically modified cells, which express the growth factor via a thermal sensitive promoter, are introduced to an area in a mammal in need of wound healing, e.g., topically or to an internal site. A device of the invention, whether external or implanted, emits heat, and the growth factor in the genetically modified donor cells is expressed in an effective amount.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention. The scope should, therefore, be determined with reference to the appended claims, along with the full scope of equivalents to which such claims are entitled.

What is claimed is:

1. A system, comprising;
a gene regulatory signal delivery device that emits a regulatory signal which directly or indirectly regulates a regulatable, field sensitive transcriptional control element; and
a controller coupled to the gene regulatory signal delivery device, the controller adapted to control the emission of the regulatory signal based on at least a sensed physiological parameter or a command,
wherein the gene regulatory signal delivery device comprises an electromagnetic field generator adapted to emit an electromagnetic field having a predetermined frequency and strength and/or comprises a thermal radiator adapted to emit a thermal energy.

2. The system of claim 1, further comprising a physiological parameter sensor coupled to the controller, wherein the sensor is adapted to sense a physiological parameter indicative of a predetermined condition.

3. The system of claim 2, wherein the predetermined condition is a cardiac condition.

4. The method of claim 3, wherein the cardiac condition is myocardial ischemia, reperfusion or remodeling.

5. The system of claim 4, further comprising a telemetry module coupled to the controller, the telemetry module adapted to receive the command.

6. The system of claim 2, further comprising an event detector to detect the predetermined condition from the sensed physiological parameter, wherein the controller is adapted to control the emission of the regulatory signal in response to a detection of the predetermined condition.

7. The system of claim 6, wherein the sensor comprises a sensor sensing a physiological parameter indicative of ischemia, and the event detector comprises an ischemia detector.

8. The system of claim 2, wherein the sensor comprises an impedance sensor to sense tissue impedance.

9. The system of claim 2, wherein the sensor comprises a metabolic sensor adapted to sense a signal indicative of a cardiac metabolic level.

10. The system of claim 9, wherein the sensor comprises at least one of a pH sensor, an oxygen pressure (PO₂) sensor, a carbon dioxide pressure (PCO₂) sensor, a glucose sensor, a creatine sensor, a C-reactive protein sensor, a creatine kinase sensor, and a creatine kinase-MB sensor.

11. A system, comprising;
an implantable medical device system including:
a sensor to sense a physiological parameter indicative of a predetermined condition;
an implant telemetry module to receive an external command;
a gene regulatory signal delivery device that emits a regulatory signal which directly or indirectly regulates a regulatable, field sensitive transcriptional control element; and
an implant controller coupled to the sensor and the implant telemetry module, the implant controller including a gene expression control module adapted to control the emission of the regulatory signal based on at least one of a sensed physiological parameter and the external command; and
an external system including an external telemetry module to transmit the external command to the implant telemetry module,
wherein the gene regulatory signal delivery device comprises an electromagnetic field generator which emits an electromagnetic field having a predetermined frequency and strength and/or comprises a thermal radiator which emits a thermal energy.

12. The system of claim 11, wherein the implantable medical device system further comprises an event detector to detect the predetermined condition from the sensed physiological parameter, and wherein the implant controller is adapted to control the emission of the regulatory signal in response to at least one of the predetermined condition or the external command.

13. The system of claim 11, wherein the sensor comprises a sensor sensing an physiological signal indicative of ischemia, and the event detector comprises an ischemia detector.

14. The system of claim 13, wherein the sensor comprises an impedance sensor to sense tissue impedance.

15. The system of claim 13, wherein the sensor comprises a metabolic sensor adapted to sense a signal indicative of a cardiac metabolic level.

16. The system of claim 13, wherein the implantable medical device system further comprises a pacing circuit coupled to the implant controller, and wherein the implant controller includes a pacing control module adapted to control a delivery of pacing pulses optionally in conjunction with the emission of the regulatory signal.

17. The system of claim 13, wherein the external system comprises:
a presentation device to present the sensed physiological parameter; and a user input device to receive the external command.

18. A method, comprising:
sensing a physiological parameter indicative of a predetermined condition;
detecting the predetermined condition from the sensed physiological parameter; and
delivering to mammalian cells a regulatory signal which directly or indirectly regulates expression from a regulatable, field sensitive transcriptional control element in the cells in response to at least the detection of the predetermined condition, wherein the regulatable, field sensitive transcriptional control element is operably linked to an open reading frame to form an expression cassette or the regulatable, field sensitive transcriptional control element is operably linked to an open reading frame in an endogenous gene,
wherein thermal energy and/or electromagnetic energy of a predetermined frequency and strength is delivered.

19. The method of claim 18, wherein the expression cassette is in a cell in a mammal.

20. The method of claim 18, wherein sensing the physiological parameter comprises sensing the physiological parameter with an implantable sensor.

21. The method of claim 20, further comprising receiving a command, and delivering the regulatory signal in response to the command.

22. The method of claim 21, wherein receiving the command comprises receiving an external command transmitted to an implantable device from an external system.

23. The method of claim 22, further comprising:
transmitting one or more of the sensed physiological parameters and a detection of the predetermined condition to an external system; and
presenting the one or more of the sensed physiological parameters and a detection of the predetermined condition through the external system.

24. The method of claim 23, wherein the sensing the physiological parameter comprises sensing a physiological parameter indicative of ischemia, and detecting the predetermined condition comprises detecting an ischemia.

25. The method of claim 18, further comprising delivering pacing pulses in conjunction with delivering the regulatory signal.

26. The method of claim 18, further comprising delivering cardioversion/defibrillation shocks in conjunction with delivering the regulatory signal.

27. A method to control expression of an open reading frame present in cells of a mammal, comprising:
providing a mammal comprising the system of claim 1 or 11, wherein the mammal comprises cells comprising an expression cassette having the regulatable, field sensitive transcriptional control element operably linked to an open reading frame or comprising an endogenous gene comprising the regulatable, field sensitive transcriptional control element operably linked to an open reading frame; and
directing signal emission in response to the sensed physiological parameter or external command so as to control expression of the open reading frame,
wherein the regulatable, field sensitive transcriptional control element is regulated by thermal energy and/or by electromagnetic energy.

28. The method of claim 27, wherein expression of the open reading frame alters nerve regeneration, bone growth or wound healing.

29. The method of claim 27, wherein the system of claim 1 or 11 is the system of claim 1 wherein the gene regulatory signal delivery device is an external device.

30. The method of claim 27, wherein expression of the open reading frame inhibits ischemia or reperfusion injury.

31. The method of claim 27, wherein the physiological parameter is sensed by an implanted sensor.

32. The method of claim 27, wherein the regulatory signal is delivered in response to a command.

33. The method of claim 32, wherein the command is an external command.

34. The method of claim 27, wherein the system is implanted in or on the heart.

35. The method of claim 27, wherein the system is implanted in or on a blood vessel.

36. The method of claim 27, wherein signal emission controls expression of at least one endogenous gene.

37. The method of claim 27, wherein signal emission controls expression of the regulatable, field sensitive transcriptional control element in the expression cassette.

38. The method of claim 18 or 27, wherein the regulatable, field sensitive transcriptional control element comprises a heat shock protein promoter.

39. The method of claim 18 or 27, wherein the open reading frame encodes a growth factor, Akt, hypoxia inducible factor, or a pacemaker ion channel.

40. The method of claim 27, wherein the cells comprising the expression cassette are exogenously administered donor cells.

41. The method of claim 27, wherein the expression cassette is introduced to the cells via recombinant virus.
